(19) United States
(12) Patent Application Publication
(10) Pub. No.: US 2008/0103462 A1
(43) Pub. Date: May 1, 2008

(54) WOUND HEALING PATCH WITH INTEGRAL PASSIVE VACUUM AND ELECTROSTIMULATION

(76) Inventors: Stuart Wenzel, San Carlos, CA (US); Mariam Maghribi, Fremont, CA (US); Mark Huang, Pleasanton, CA (US); Zara Sieh, Pleasanton, CA (US); Michael V. Williamson, Clayton, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/876,175

(22) Filed: Oct. 22, 2007

Related U.S. Application Data
(60) Provisional application No. 60/863,425, filed on Oct. 30, 2006.

(51) Int. Cl. A61M 35/00

(52) U.S. Cl. 604/313

(57) ABSTRACT

In one example, the present invention is directed to wound-healing patche that passively or actively draw fluids from a wound using an internal, integral vacuum source. The patches may also include electrodes and electronics for electrostimulation, and bioactive compounds that promote healing, such as anti-inflammatory agents.

### CROSS-REFERENCE

This application claims priority from Provisional Application No. 60/863,425 filed Oct. 30, 2006, entitled Wound Healing Dressing with Integral Passive Vacuum and Electrostimulation which application is fully incorporated herein by reference.

The present invention is directed to a wound healing patch and, more particularly, to an improved wound healing patch incorporating an integral passive vacuum system to draw fluids from the wound and assist in wound healing.

### BACKGROUND OF THE INVENTION

Wounds and their complications are a major problem in both hospital and home settings. Healing such wounds is a priority for those who work in the health care field. There are many types of wounds that have different associated complications. For example, diabetic ulcers are caused and exacerbated by poor blood flow and inflammation, and are slow to heal, or may never heal if left untreated. This can lead to infection and scarring, among other problems. Thus, devices that promote wound healing are highly beneficial. While band aids and other wound dressings assist in the healing process by protecting the wound and helping to absorb fluids, it would be beneficial to have a wound healing patch which actively promotes the healing process.

### SUMMARY OF THE INVENTION

The present invention is directed to wound-healing patches that passively or actively draw fluids from a wound using an internal, integral vacuum source. The patches may also include electrodes and electronics for electrostimulation, and bioactive compounds that promote healing, such as anti-inflammatory agents.

The present invention is further directed to a method of treating wounds using a novel wound-healing patch including an internal, integral vacuum source, wherein fluids are withdrawn from the wound by the vacuum source to promote wound healing.

The present invention is further directed to a method of treating wounds using a novel wound-healing patch including an internal, integral vacuum source and electrostimulation circuitry, wherein fluids are withdrawn from the wound by the vacuum source while the electrostimulation circuitry is activated to promote wound healing.

The present invention is further directed to a method of treating wounds using a novel wound-healing patch including an internal, integral vacuum source and bioactive compounds, wherein fluids are withdrawn from the wound by the vacuum source, while bioactive compounds migrate from the patch into the wound and promote wound healing.

### BRIEF DESCRIPTION OF THE FIGURES

The invention will now be described, by way of example only, with reference to the following figures. The accompanying drawings, which are incorporated herein and constitute part of this specification, illustrate presently preferred embodiments of the invention, and, together with the general description given above and the detailed description given below, serve to explain features of the invention, in which:

FIGS. 1A-1D illustrate an embodiment of an apparatus and method according to the present invention, wherein a compressible foam is used to draw fluids (e.g. exudate) out of a wound.

FIGS. 2A-2B illustrate an embodiment of an apparatus and method according to the present invention where absorbent layers used to generate a passive vacuum that have individual expandable chambers isolated from each other.

FIGS. 3A-3B illustrate an embodiment of an apparatus and method according to the present invention where wall deformation and volume changes in an elastomeric structure create an internal vacuum.

FIG. 4 illustrates an embodiment of the present invention wherein discrete springs generate a vacuum force and suction.

FIGS. 5A-5C illustrate an embodiment of the present invention wherein a dissolvable sealant is used over the absorbent layer.

### DETAILED DESCRIPTION OF THE FIGURES

The following detailed description should be read with reference to the drawings, in which like elements in different drawings are identically numbered. The drawings, which are not necessarily to scale, depict selected exemplary embodiments for the purpose of explanation only and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. In addition, as used herein, the terms “patient”, “host” and “subject” refer to any human or animal subject and are not intended to limit the systems or methods to human use, although use of the subject invention in a human patient represents a preferred embodiment.

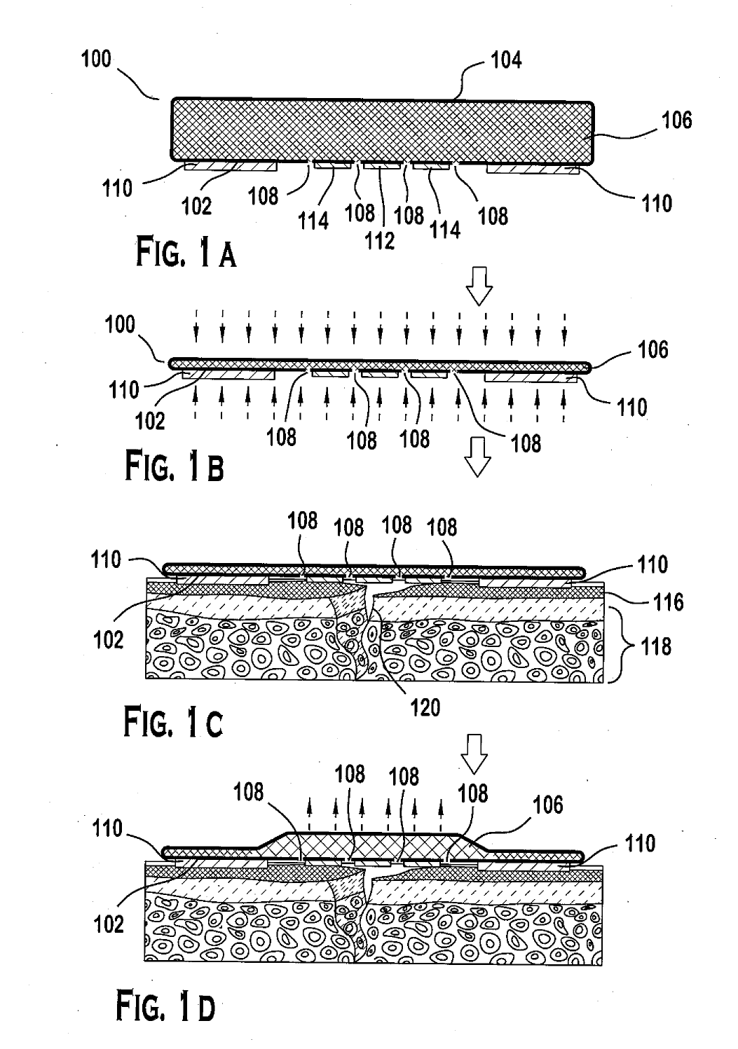

FIGS. 1A-1D illustrate an embodiment of the present invention. In the embodiment illustrated in FIGS. 1A-1D, passive vacuum, electrostimulation, and/or bioactive compounds can be used to treat wounds. Patch 100 has an internal, integrated source of negative pressure that is activated when the dressing is applied. In an alternative embodiment of the invention, the source of negative pressure may be located in close proximity to the patch, in the form of a vacuum vial or syringe that can be operated manually.

As illustrated in FIGS. 1A-1D, patch 100 includes a self-expanding, flexible absorbent material 106, which is encased in vapor-impermeable outer barrier 104 and includes inner surface 102. Outer barrier 104 completely surrounds absorbent material 106. Inner surface 102 is typically placed in contact with skin 116, and includes openings 108. In FIG. 1A, patch 100 is in an expanded state, and is unused. In FIG. 1B, patch 100 has been compressed, expelling air from absorbent material 106, preparing it for use. In FIG. 1C, patch 100 is positioned over wound 120, and attached to skin 116 by adhesive 110 or other suitable means, which is located around the periphery of the patch. Patch 100 is flexible, so it can conform to skin 116, tissue 118, and wound 120. In FIG. 1D, patch 100 is released, allowing absorbent material 106 to expand, and sucking fluids (e.g. wound exudates) through openings 108 into absorbent material 106. Patch 100 also includes wound electrode 112 and return electrodes 114, for use in electrostimulated wound healing.

In one embodiment of the present invention, patch 100 is used in a variety of ways. For example, in a first step, patch 100 is removed from external packaging in the state illustrated in FIG. 1A. In FIG. 1A, openings 108 are clear and absorbent material 106 is decompressed. In a next step, as illustrated in FIG. 1B, absorbent material 106 is compressed, reducing its overall volume, and expelling air from absorbent material 106 through openings 108. In a next step, as illustrated in FIG. 1C, patch 100 is applied to skin 116, aligning openings 108 with wound 120. Alignment of openings 108 with wound 120 allows flow of wound exudates from wound 120, through openings 108, and into absorbent material 106. In the step illustrated in FIG. 1C, patch 100 is fastened to skin 116 by adhesive 110. In a next step, as illustrated in FIG. 1D, patch 100 is released, allowing absorbent material 106 to decompress, increasing its overall volume. As the volume of absorbent material 106 increases, a slight vacuum (or negative pressure) is created in absorbent material 106. The slight vacuum in absorbent material 106 provides a driving force for flow of wound exudates from wound 120 into absorbent material 106. Finally, patch 100 is replaced, as needed, until wound 120 has healed.

In embodiments of the present invention, patch 100 may be packaged with absorbent material 106 in a compressed state. Patch 100 is removed from its package, and secured to skin 116 with adhesive 110. Adhesive 110 can be a pressure sensitive adhesive, for example. After patch 110 is fastened to skin 116, absorbent material 106 expands, creating suction that draws fluid from wound 120, through openings 108, and into absorbent material 106. Outer barrier 104 prevents air from being drawn into absorbent material 106 from areas other than openings 108, and helps to create a single path (via openings 108) for fluid flowing into absorbent material 106.

In the embodiment of the present invention illustrated in FIG. 1, self-expanding absorbent material 106 may be made using open-cell foam that is compressed prior to placing patch 100 on skin 116. Self-expanding absorbent material 106 may be compressed using an external vacuum source, such as a pump or a syringe, or may be compressed using externally applied force, such as squeezing it between fingers. Open-cell foam can be coated, for example with a flexible polymer, or in other ways treated to create outer barrier 104. Openings 108 can be included in inner surface 102 to allow fluid entry into absorbent material 106. Adhesive 110 provides an airtight seal around between the periphery of patch 100 and wound 120. In other embodiments of the present invention, an airtight seal around the periphery of patch 100 can be formed using a gasket with individual suction ports that create a vacuum seal prior to the activation of expanding absorbent material 106.

Open or closed cell foams can have high moduli (>10⁶ Pa=7600 mmHg) and can be compressed significantly (e.g., to <20% of their original volume), enabling significant pressure-volume changes when used in the present invention. For example, a foam dressing 10 cm×10 cm in area and 1 cm thick has a volume of 100 ml when expanded, but only 20 ml when compressed. This would allow up to 80 ml of fluid to enter absorbent material 106 when it is decompressed.

In an alternative embodiment of the patch illustrated in FIG. 1, a strategy is implemented that allows the patch to be moved from its package to the wound without expansion of absorbent material 106. In this embodiment, absorbent material 106 is compressed to a thin sheet while wet, and then it is dried and incorporated into patch 100. Absorbent material 106 will not generate an expansive force, or vacuum, until exposed to moisture from the wound exudates, at which time it becomes moist and expansive, generating appropriate vacuum.

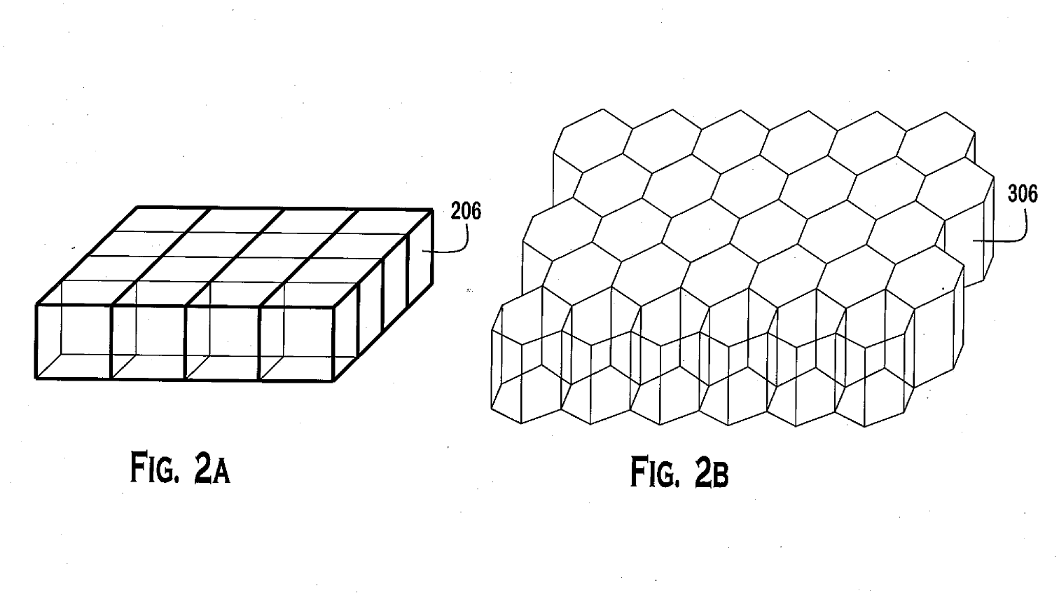

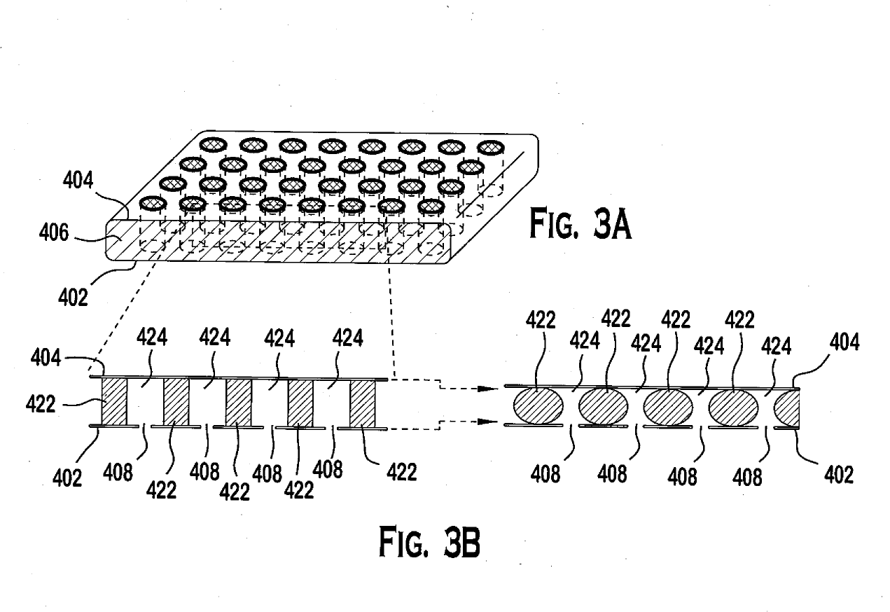

In further embodiments of the present invention, illustrated in FIGS. 2A and 2B, the inside of absorbent materials 206 and 306 can have individual expandable chambers that are isolated from each other. The chambers may be substantially empty or be filled with additional absorptive materials. In many embodiments of the present invention, the force that causes chamber expansion, and resulting suction, is relaxation of deformed chamber walls. Chamber walls can be made of resilient materials, such as elastomers. FIGS. 3A and 3B illustrate how deformation of wall 422 causes volume changes in pocket 424. Due to its elastomeric properties, wall 422 changes shape when compressed and decompressed. When it is compressed, before application to skin, the volume of pocket 424 is small. When sealed to the skin and expanded, the volume of pocket 424 increases, creating less than atmospheric pressure (a vacuum) in pocket 424. The vacuum created in pocket 424 can be used to induce flow of fluids from a wound through openings 408 and into absorbent material 406. FIG. 3A illustrates absorbent material 406 in an uncompressed state. Absorbent material 406 includes outer barrier 404. Inner surface 402 makes direct contact with skin, when in use, while outer barrier 404 forms the outer surface of absorbent material 406. In FIG. 3B, absorbent material 406 is shown in an uncompressed state on the left, and in a compressed state on the right. Absorbent material 406 includes inner surface 402 with openings 408, and outer barrier 404. Absorbent material 406 also includes walls 422, which deform when absorbent material 406 is compressed.

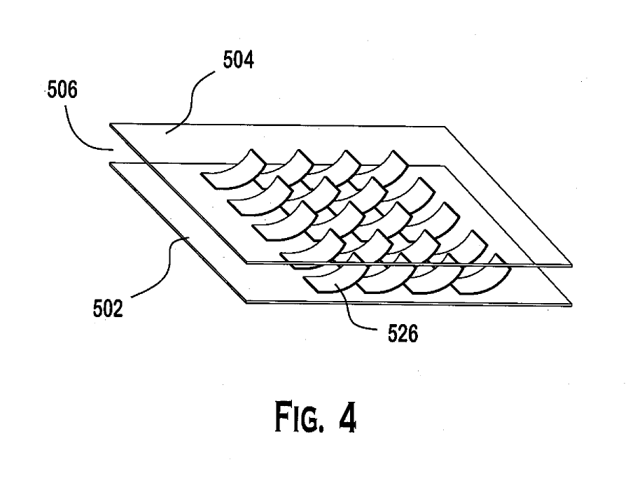

FIG. 4 illustrates an embodiment of the invention where discrete springs generate force and suction. In this design absorbent material 506 includes inner surface 502, outer barrier 504, and springs 526. Springs 526 may be made of any suitable material including spring metals (steel, beryllium copper, etc), shape memory alloys and super elastic materials (nitinol, etc) or plastics. Super elastic materials may be especially useful because of their ability to generate nearly constant force-versus-deflection in spring structures, which will result in constant force as absorbent material 506 expands and collects wound exudates. In some embodiments of the present invention, shape-memory materials can be used to modify the shape of springs 526, by heat cycling between martensitic and austenitic states of the material. Heat can be supplied by the body or by electrical means such as current flow.

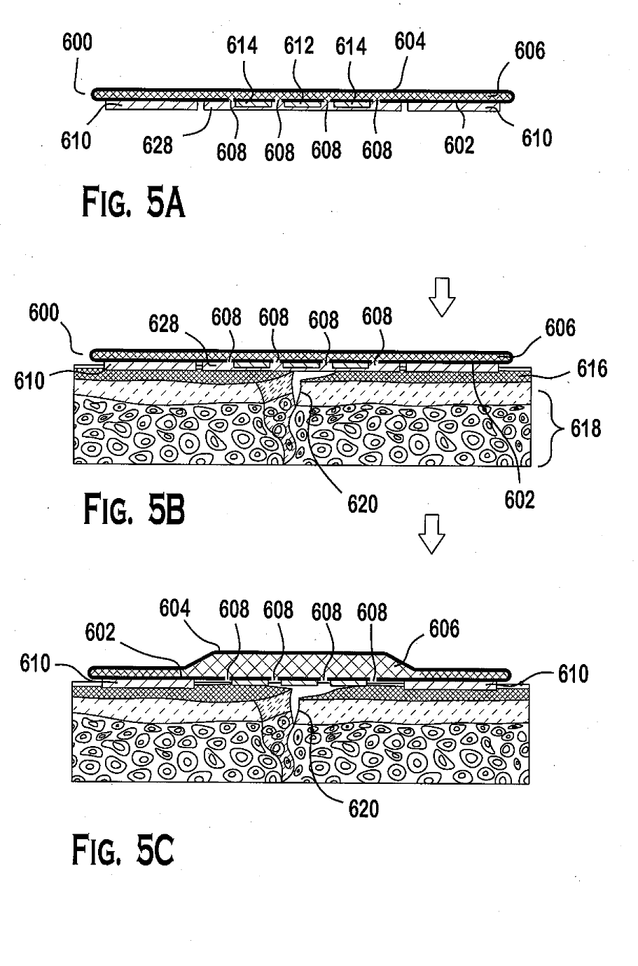

FIGS. 5A-5C illustrate a further embodiment of the present invention, wherein dissolvable sealant 628 may be included. Dissolvable sealant 628 covers and seals openings 608 until exposed to wound exudates. Wound exudates dissolve sealant 628, clearing a path through openings 608. Once openings 608 are clear, absorbent material 606 can expand, creating suction and transferring exudates from wound 620 into absorbent material 606. Sealant 628 may be dissolved through a number of mechanisms, that may be triggered by moisture, pressure, temperature, and/or radiation (including ulkaviolet, optical and infrared). Sealant 628 may be constructed of, for example, starch, sugar, PVP, PEG, PEO, PVA, and chitin. Sealant 628 may be dissolved electronically once patch 600 is applied. Voltage or current may be applied to sealant 628, triggering dissolution of sealant 628 and clearing openings 608. In FIG. 5A, patch 600 in an initial state. Absorbent material 606 is compressed, and openings 608 are plugged with sealant 628. In FIG. 5B, patch 600 has been applied to skin 616 and wound 620. Adhesive 610 bonds patch 600 to skin 616, and forms an airtight seal around the periphery of patch 600. In FIG. 5C, exudates from wound 620 have dissolved sealant 628, clearing openings 608. Once openings 608 are clear of sealant 628, absorbent material 606 expands, drawing exudates through openings 608. Patch 600 also includes wound electrodes 612 and return electrodes 614, for use in electrostimulated wound healing.

In embodiments of the present invention, patch 600 is used in a variety of ways. For example, in a first step, patch 600 is removed from external packaging in the state illustrated in FIG. 5A. In FIG. 5A, openings 608 are blocked by sealant 628 and absorbent material 606 is compressed. In a next step, as illustrated in FIG. 5B, patch 600 is applied to skin 616, aligning openings 608 with wound 620. Alignment of openings 608 with wound 620 allows flow of wound exudates from wound 620, through openings 608, and into absorbent material 606. In the step illustrated in FIG. 5B, patch 600 is fastened to skin 616 by adhesive 610. In a next step, as illustrated in FIG. 5C, patch 600 is released and sealant 628 dissolves, allowing absorbent material 606 to decompress, increasing its overall volume. As the volume of absorbent material 606 increases, a slight vacuum (or negative pressure) is created in absorbent material 606. The slight vacuum in absorbent material 606 provides a driving force for flow of wound exudates from wound 620 into absorbent material 606. As mentioned previously, sealant 628 is dissolved by moisture in exudates from wound 620. Finally, patch 600 is replaced, as needed, until wound 120 has healed.

In a further embodiment of the present invention, wound-healing patches can include bioresorbable scaffold matrixes with incorporated bioactive agents, such as anti-inflammatory agents (i.e. omega-3 fatty acids, eicosanoids, prostaglandin E1), collagen, keratinocytes, and fibroblasts, which aid the wound-healing process.

In further embodiments of the present invention, mechanical and electromechanical means can be used to activate absorbent material. Mechanical and electromechanical means include breakable seals or valves that are actuated by the user, or electronically. Absorbent material may also be activated manually, by removal of a membrane after the patch has been placed on a wound. In some embodiments, a shutter or gasket like device may be used to seal the gap left by the membrane.

In further embodiments of the present invention, electronics and electrodes are used to electrostimulate wound tissue. Patches may include electronics and electrodes to enable electrostimulation and other functions that may enhance wound healing. In addition, electronics and electrodes can display and transmit data to the patient or health-care professional. Embodiments of the present invention may include: batteries (e.g., thin-film lithium or coin cells); passive components (e.g., resistors, capacitors, inductors); active chips (e.g., transistors, microprocessors, memory, wireless transceivers); conductive, resistive and insulating layers and traces (e.g., conductive inks and/or sputtered metal layers that may be spatially patterned); heaters and thermoelectric coolers; or materials to generate galvanic voltage and current to facilitate wound healing, such as zinc and silver.

In other embodiments of the present invention, bioactive compounds that promote wound healing may be included in the patch. Bioactive compounds useable in the present invention include: anti-microbial agents and anti-inflammatory agents such as NSAIDs (non-steroidal anti-inflammatory agents), cortical steroids, omega-3 fatty acids and other eicosanoid modulators, anti-inflammatory eicosanoids (e.g., prostaglandin E1), and cytokine inhibitors such as Remicade (an anti-TNF drug), etc.

In other embodiments of the present invention, an oxygen pump can be connected to a wound-healing patch, providing hyperbaric oxygen treatment to enhance wound healing and minimize bacterial infection.

In embodiments of the present invention, the combination of suction creating absorbent material and electrostimulation in a single disposable device provides means for improved wound healing. Moreover, the size and simplicity of the design enhances at-home treatment of chronic wounds by patients who may otherwise require outpatient or clinical procedures.

While preferred embodiments of the present invention have been shown and described herein, it will be obvious to those skilled in the art that such embodiments are provided by way of example only. Numerous variations, changes, and substitutions will now occur to those skilled in the art without departing from the invention. Various alternatives to the embodiments of the invention described herein may be employed in practicing the invention. It is intended that the following claims define the scope of the invention and that methods and structures within the scope of these claims and their equivalents be covered thereby.

What is claimed is:

1. A non-invasive wound-healing device comprising:.
an integrated source of negative pressure; and,
electronics suitable to implement electrostimulation and other functions that may assist wound healing.

2. The wound-healing device according to claim 1, wherein the electronics comprise electrostimulation circuitry.

3. The wound healing device of claim 2, wherein the electrostimulation circuitry comprises batteries; passive components; conductive, resistive and insulating layers or traces; heaters; thermoelectric coolers; or materials to generate galvanic voltage or current.

4. The wound healing device of claim 1, wherein the electronics are capable of implementing other functions that may assist wound healing.

5. The wound healing device of claim 4, wherein the electronics are suitable to display or transmit data.

6. The wound healing device of claim 5, wherein the electronics comprise active chips such as transistors, microprocessors, memory or wireless transceivers.

7. The wound healing device of claim 1, wherein the source of negative pressure comprises a pre-existing evacuated volume within the wound-healing device.

8. The wound-healing device of claim 7, wherein the pre-existing evacuated volume comprises a self-expanding, flexible material or composite.

9. The wound-healing device of claim 8, wherein the self-expanding material comprises compressed open cell foam.

10. The wound healing device of claim 9, wherein the compressed open cell foam is coated or treated to make a vacuum seal.

11. The wound healing device of claim 7, wherein the pre-existing evacuated volume comprises individual expanding chambers that are isolated and sealed from each other.

12. The wound healing device of claim 11, wherein the chambers comprise an absorptive or adsorptive material.

13. The wound healing device of claim 7, wherein the pre-existing evacuated volume comprises one or more springs.

14. The wound healing device of claim 13, wherein the springs comprise metal, shape memory alloys, superelastic materials, plastics or a combination of these.

15. The wound healing device of claim 10, wherein the coating or treatment is removable or dissolvable.

16. The wound healing device of claim 15, wherein the coating comprises starch, sugar, PVP, PEG, PEO, PVA, chitin or combinations of these.

17. The wound healing device of claim 15, wherein the coating is electrochemically dissolvable.

18. The wound healing device of claim 15, wherein the coating comprises a bioresorbable matrix.

19. The wound healing device of claim 18, wherein the coating comprises one or more bioactives incorporated into the bioresorbable matrix.

20. The wound healing device of claim 19, wherein the one or more bioactives comprise anti-inflammatory agents, antimicrobial agents, cortical steroids, eicosanoid modulators, antiflammatory eicosanoids, cytokine inhibitors, collagen, keratinocytes, fibroblasts or combinations of these.

21. The wound healing device of claim 1, wherein a wearable oxygen pump is operatively disposed relative to the device.

22. A method of treating a wound using a wound healing patch, said method comprising the steps of:
using said patch to generate a passive vacuum which draws fluid from said wound site into said wound healing patch;
passing electrical current through said wound using electrodes on said patch.

23. A method according to claim 22 wherein said method further includes the steps of injecting bioactive compounds into said wound.

24. A method according to claim 23 wherein said bioactive compounds comprise agents which promote healing or reduce inflammation.
